(19) United States
(12) Patent Application Publication
(10) Pub. No.: US 2004/0191802 A1
(43) Pub. Date: Sep. 30, 2004

(54) Phospholamban polymorphism and methods of assessment

(76) Inventors: Evangelia G. Kranias, Cincinnati, OH (US); Kobra Haghighi, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/691,412

(22) Filed: Oct. 22, 2003

Related U.S. Application Data
(60) Provisional application No. 60/420,295, filed on Oct. 22, 2002.

(51) Int. Cl. C12Q 1/68

(52) U.S. Cl. 435/6

(57) ABSTRACT

Methods of phospholamban mutation/polymorphism assessment in an individual comprise the steps of obtaining a sample from an individual; isolating a nucleotide fragment containing a phospholamban coding region from the sample; analyzing the nucleotide fragment; and comparing the analysis of the nucleotide fragment with a predetermined phospholamban nucleotide fragment sequence to determine whether the individual carries a phospholamban polymorphism. An isolated phospholamban mutation/polymorphism fragment comprises SEQ ID NO:1. Methods for determining if an individual is at risk for developing a cardiovascular disease are also provided.

### RELATED APPLICATION

This application claims priority under 35 U.S.C. §119 of U.S. Provisional Application Serial No. 60/420,295 filed Oct. 22, 2002.

### GOVERNMENT INTERESTS

This invention was made, at least in part, with funds from the Federal Government, awarded through grant numbers HL52318 and HL26057. The U.S. Government therefore has certain acknowledged rights to the invention.

### FILED OF THE INVENTION

The present invention is directed towards methods of phospholamban (PLN) polymorphism assessment in an individual and methods for determining if an individual is at risk for developing a cardiovascular disease. The invention is also directed toward an isolated phospholamban polymorphism (mutation) fragment.

### BACKGROUND OF THE INVENTION

Heart failure, a condition characterized by the impaired ability of the heart to adequately supply oxygen and nutrient-rich blood to the entire body, is a major cause of death and disability. This form of cardiovascular disease is increasing in incidence and prevalence, with nearly 400,000 new cases annually. It typically carries an overall five-year mortality of 50% with the primary cure being cardiac transplantation. The causes of heart failure include, but are not limited to, coronary artery disease, congenital structural defects, endocarditis, and cardiomyopathies. While many of these triggers for heart failure have been thoroughly described and analyzed, the basic mechanisms behind the pathogenicity of dilated cardiomyopathy (DCM) remain largely unresolved. Coincidently, despite advances in medical therapy, DCM continues to have high rates of morbidity and mortality. DCM is difficult to classify, as its pathology varies; yet it is typified by an increase in ventricular chamber volume, increased cardiac systolic and diastolic dimensions and resultant progression into heart failure.

Studies have indicated that as many as 35% of DCM cases may be familial. These studies have stimulated efforts to discover molecular genetic defects that cause DCM. In recent years, mutations in genes encoding contractile, cytoskeletal, nuclear membrane, and other proteins have been associated with familial and sporadic DCM. Although these reports clearly establish DCM as a genetically heterogeneous disorder, the alterations in the molecular and cellular mechanisms leading to DCM as a result of these mutations remain poorly delineated. Owing to the increasing incidence and prevalence of heart failure, it would be advantageous to accurately assess molecular genetic defects that cause heart failure in an individual.

### SUMMARY OF THE INVENTION

Accordingly, it is an object of the invention to provide methods of phospholamban polymorphism assessment in an individual. It is a further object of the invention to provide an isolated phospholamban polymorphism fragment. It is yet a further objection of the invention to provide methods for determining if an individual is at risk for developing a cardiovascular disease

In accordance with one aspect of the invention, methods of phospholamban polymorphism assessment in an individual are provided. The method comprises the steps of obtaining a sample from the individual; isolating a nucleotide fragment containing a phospholamban coding region from the sample; analyzing the nucleotide fragment; and comparing the analysis of the nucleotide fragment with a predetermined phospholamban nucleotide fragment sequence to determine whether the individual carriers a phospholamban polymorphism.

In accordance with another aspect of the invention, there is provided an isolated phospholamban polymorphism fragment.

In accordance with yet another aspect of the invention, there is provided a method for determining if an individual is at risk for developing a cardiovascular disease. The method comprises obtaining a sample from the individual; isolating a nucleotide fragment containing a phospholamban coding region from the sample; analyzing the nucleotide fragment; and comparing the analysis of the nucleotide fragment with a predetermined phospholamban nucleotide fragment sequence to determine whether the individual carries a phospholamban polymorphism characteristic of an increased risk of developing cardiovascular disease.

In accordance with yet another aspect of the invention, there is provided a method for determining if an individual is at risk for developing a cardiovascular disease. The method comprises analyzing a sample from the individual to determine the presence of a phospholamban polymorphism characteristic of an increased risk of developing cardiovascular disease.

The present invention is advantageous for assessing phospholamban polymorphism in an individual. The present invention is also advantageous for determining if an individual is at risk for developing a cardiovascular disease. Additional embodiments, objects and advantages of the invention will become more fully apparent in view of the following detailed description.

### DETAILED DESCRIPTION OF THE DRAWINGS

The following detailed description will be more fully understood in view of the drawings comprising:

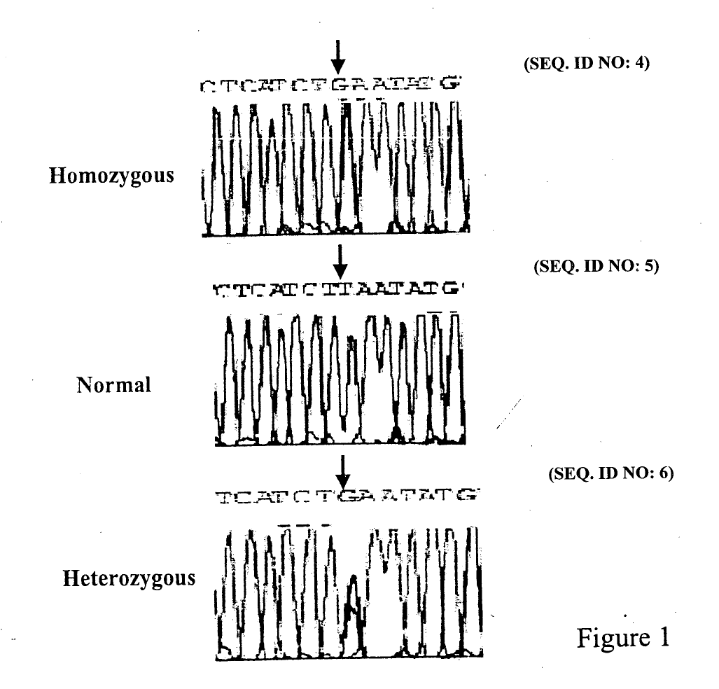

FIG. 1 is a graphical illustration of a human phospholamban gene mutation in the homozygous, heterozygous and normal form;

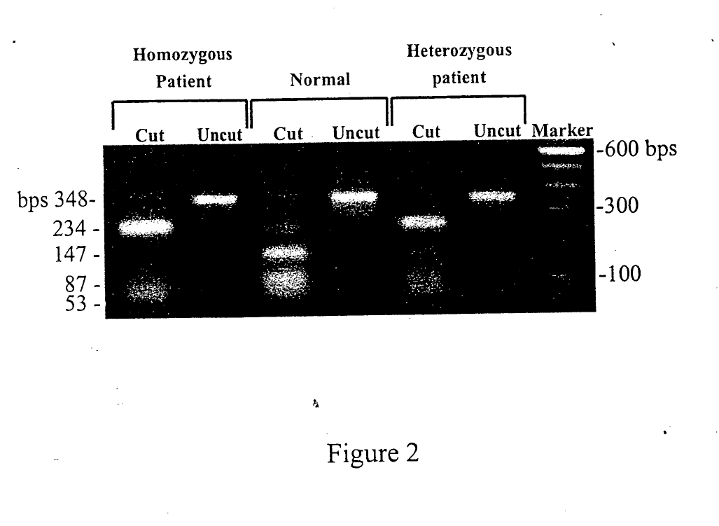

FIG. 2 depicts restriction endonuclease digestion patterns of Polymerase Chain Reaction (PCR) amplified DNA fragments from normal individuals and individuals with a phospholamban polymorphism;

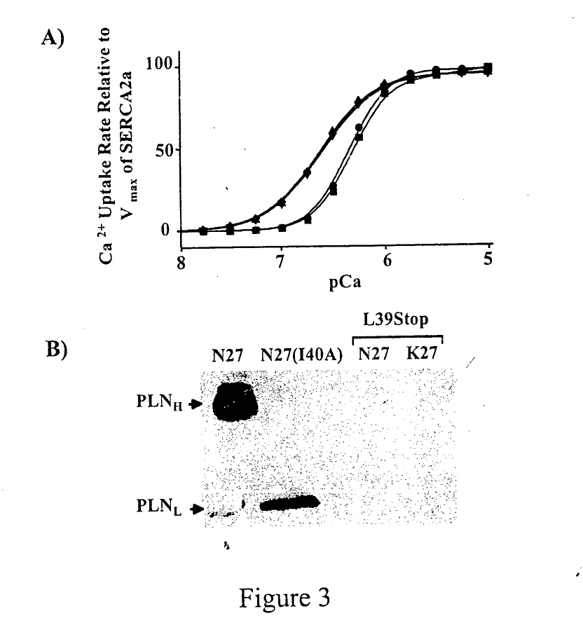

FIG. 3 is an illustration of the effect of wild type and mutant phospholamban on the Ca²⁺ affinity of SERCA2a;

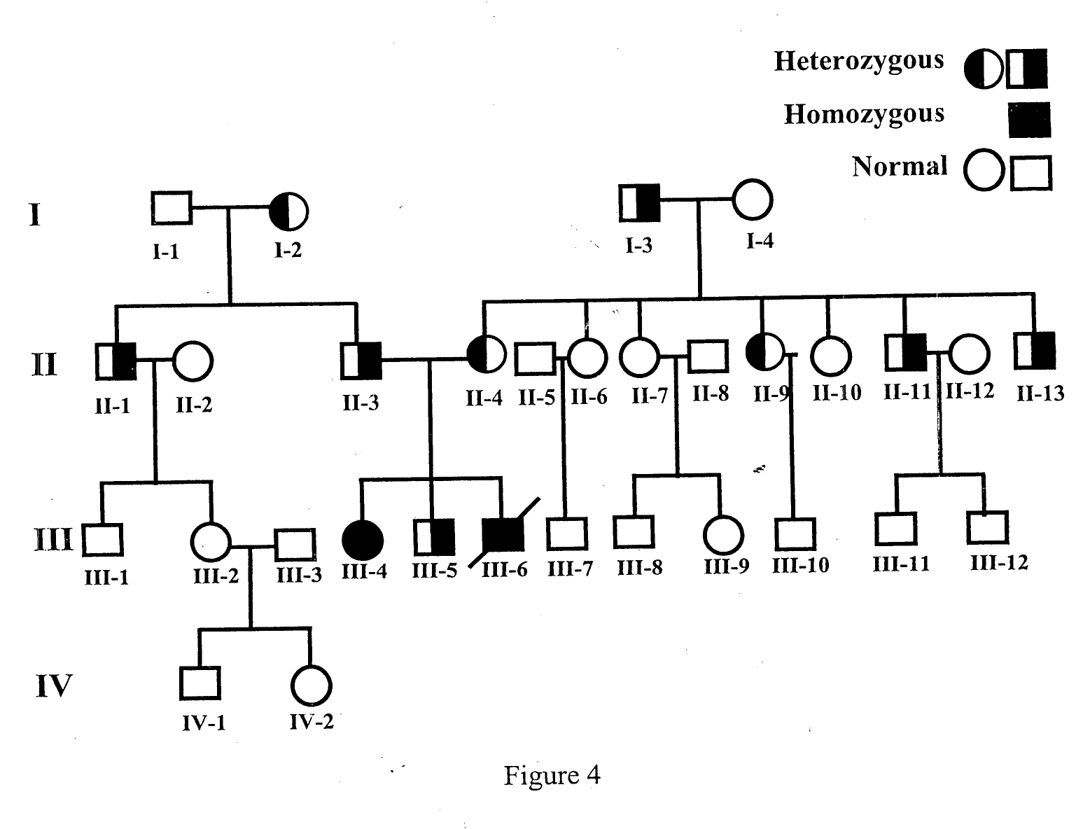

FIG. 4 is a pedigree analysis of a Greek dilated cardiomyopathy (DCM) family analyzed for the presence or absence of the T116 to G mutation in the coding sequence of the phospholamban (PLB) gene;

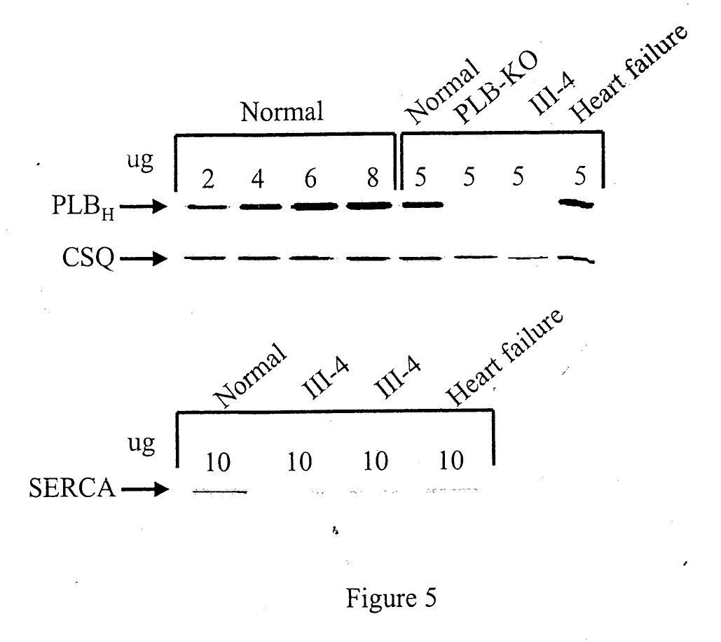

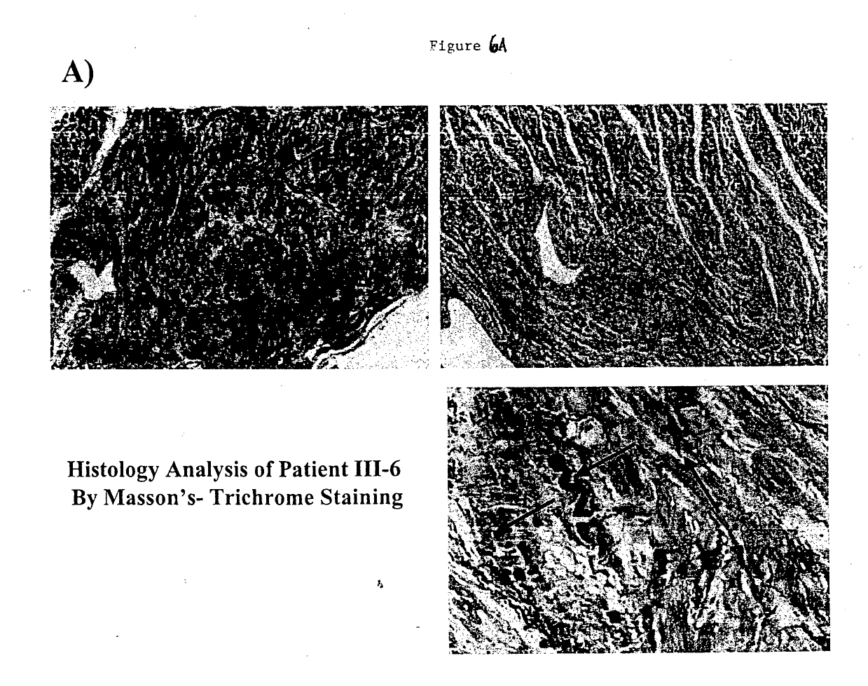

FIG. 5 is an illustration of quantitative immunoblotting of phospholamban and SERCA2a from explanted heart tissue of homozygous proband III-4; and

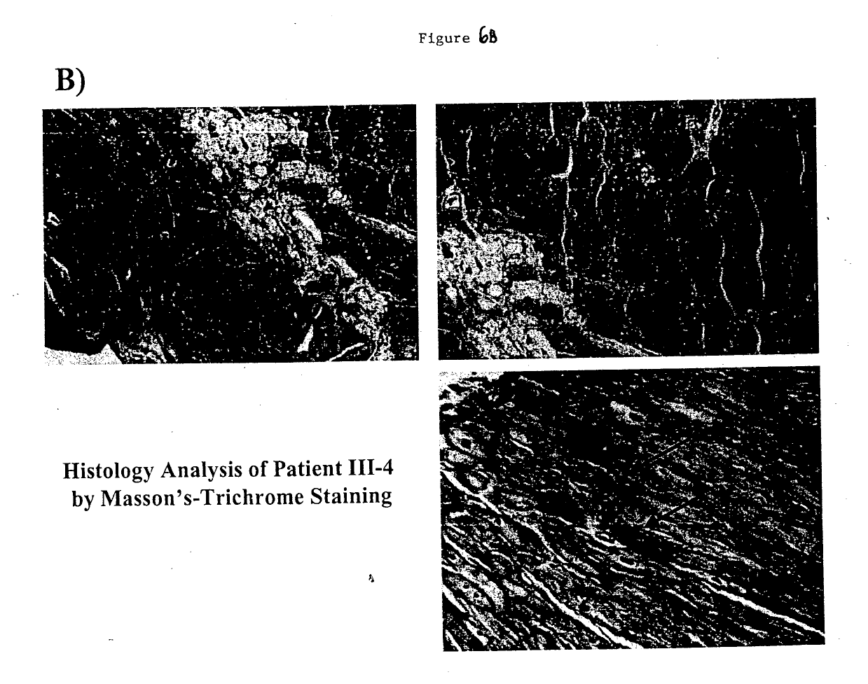

FIGS. 6A-B are illustrations of histological analysis of hearts from patients homozygous for the T116 to G mutation in the phospholamban gene.

### DETAILED DESCRIPTION OF THE INVENTION

Heart failure is a major cause of death and disability. The causes of heart failure include, but are not limited to, coronary artery disease, congenital structural defects, endocarditis, and cardiomyopathies. The heart's fine pumping action is highly regulated by the calcium levels in cardiac cells. Calcium itself is mainly regulated by the sarcoplasmic recticulum (SR) an internal membrane system in the cardiac cells. During contraction, calcium is released from the SR via ryanodine receptors and during relaxation, calcium is resequestered into the SR by the SR Ca²⁺-ATPase (SERCA). The active reuptake of Ca2⁺ into the SR is under reversible regulation by phospholamban.

One of the major characteristics of the progression to heart failure observed in DCM is impaired diastolic function. This suggests a depressed ability of the sarcoplasmic reticulum (SR) to sequester enough Ca²⁺ from the cytosol to allow for sufficient cardiac muscle relaxation. The sequestration of Ca²⁺ into the SR is mediated by the Ca²⁺-ATPase (SERCA2a). In cardiac muscle, the affinity of this enzyme for Ca²⁺ is regulated by phospholamban. Phospholamban is a critical regulator of myocardial contractility and the heart's responses to β-agonists and mutant forms of SERCA or phospholamban may result in drastic changes in SR Ca²⁺ transport activity. These alterations are associated with impaired myocardial function leading to cardiac remolding.

Abnormal Ca²⁺ handling has been implicated as a major underlying mechanism for diastolic dysfunction in patients with end stage heart failure. Studies in myocytes from patients with end stage diastolic heart failure demonstrated relatively normal Ca²⁺ kinetics in systole, coupled with delays in clearance of Ca²⁺ from the myoplasm during diastole. The delay in removal of Ca²⁺ from the contractile apparatus and cytosol contributes to intracellular Ca²⁺ overload in diastole and may underline the prolonged isovolumic relaxation and impaired early ventricular filling.

Phospholamban is a low-molecular weight integral SR membrane phospho protein which in its dephosphorylated state is an inhibitor of the SERCA activity. While phospholation of phospholamban relieves this inhibition, this protein has been identified as a key regulator of myocardial contractility. There exists an inverse relationship between myocardial contractility and the levels of phospholamban. Thus, any process which alters the levels or activity of phospholamban will result in altered myocardial contractility. Therefore, while not wishing to be bound by theory, the inventors believe that germ line mutations in phospholamban may contribute to diminution in cardiac function observed in late stage heart failure.

The inventors have discovered that a mutant amino acid, Leu³⁹, is located in the transmembrane domain of the 52 amino acid phospholamban protein. This domain is highly conserved among species. While not wishing to be bound by theory, it is proposed to be involved in the regulatory effects of phospholamban on the affinity of SERCA2a for Ca²⁺. Using alanine-scanning mutagenesis, the inventors have discovered that some mutations in this domain are associated with increased inhibition while others resulted in loss of the inhibitory effects on the SR Ca²⁺-pump.

Moreover, as detailed in the examples of the present invention, the inventors have also shown that the mutation of Leu39 to a stop codon in the human phospholamban gene codes for a truncated phospholamban protein, which is not present in sufficient to inhibit the Ca²⁺-pump. While not wishing to be bound by theory, the inventors believe that this lack of inhibition may be associated with abnormal handling of intracellular Ca²⁺ and consequently, impaired contractility in end stage heart failure.

Mutations in other cardiac genes have also been described to exist in heart failure. Moreover, the literature on the role of phospholamban in heart failure is contradictory. Some studies reported reduction, while others observed no alteration in phospholamban levels in human heart failure. The inventors have discovered that, at least in some patients, the uninhibited SR Ca²⁺-pump activity may not be beneficial. These findings by the inventors are the first genetic evidence that a mutation in the phospholamban gene-coding region may contribute to the clinical phenotype of human heart failure.

Accordingly, the inventors have discovered a phospholamban polymorphism fragment. In one embodiment, the present invention is directed towards methods for phospholamban polymorphism assessment in an individual. The methods comprise the steps of obtaining a sample from an individual; isolating a nucleotide fragment containing a phospholamban coding region from the sample; analyzing the nucleotide fragment; and comparing the analysis of the nucleotide fragment with a predetermined phospholamban nucleotide fragment sequence to determine whether the individual carries a phospholamban polymorphism. In another embodiment, the invention is directed toward a phospholamban polymorphism fragment.

As used herein, “individual” is intended to refer to an animal, including but not limited to mammals, including humans. As used herein, a phospholamban polymorphism fragment comprises the nucleic acid sequence:

| | |
|---|---|
| ATGGAGAAAGTCCAATACCTCACTCGCTCAGCTATAAGAAGAGCCTCAAC | (SEQ ID NO: 1) |
| CATTGAAATGCCTCAACAAGCACGTCAAAAGCTACAGAATCTATTTATCA | |
| ATTTCTGTCTCATCTTAATATGTCTCTTGCTGATCTGTATCATCGTGATG | |
| CTTCTCTGA. | |

One skilled in the art will appreciate the various known samples for obtaining a nucleotide fragment containing a phospholamban coding region. Samples include, but are not limited to blood samples, tissue samples, or combinations thereof. Moreover, one skilled in the art will appreciate the various known techniques for isolating a nucleotide fragment containing a phospholamban coding region from the sample, any of which may be used herein. Furthermore, one skilled in the art will appreciate the various known techniques for analyzing a nucleotide fragment, any of which may be used herein. In one embodiment, analyzing a nucleotide fragment comprises the steps of amplifying a nucleotide fragment, purifying the amplified nucleotide fragment and sequencing the purified nucleotide fragment. In another embodiment, analyzing a nucleotide fragment comprises the steps of amplifying a nucleotide fragment, purifying the amplified nucleotide fragment and subjecting the amplified nucleotide fragment to restriction endonucleose enzyme analysis.

As used herein, “predetermined” is intended to refer to a normal or control phospholamban nucleotide fragment sequence. As used herein “heterozygous” is intended to refer to a cell and/or organism having two different alleles of a particular gene. In one embodiment, the heterozygous form of the phospholamban polymorphism comprises a change in the nucleotide acid 116 from nucleic acid T to nucleic acid G and further, a change in codon 39 from a Leucine codon to a stop codon in one allele. As used herein “homozygous” is intended to refer to a cell and/or organism having two identical alleles of a particular gene. In one embodiment, the homozygous form of the phospholamban polymorphism comprises a change in the nucleotide acid 116 from nucleic acid T to nucleic acid G and further, a change in codon 39 from a Leucine codon to a stop codon in both alleles. In a further embodiment, the phospholamban polymorphism comprises removal of a restriction endonuclease site.

The inventors have discovered that patients carrying the phospholamban polymorphism in the homozygous form have early onset of the disease and cardiac failure. However, heterozygous individuals, carrying one normal and one defected phospholamban alleles, are mostly asymptomatic (some with mildly hypertrophy characteristics), and exhibit no cardiac dysfunction under normal and stress echocardiography. While not wishing to be bound by theory, the inventors believe the different phenotypes observed in patients, indicate that there may be a gene dosage dependency. Cardiovascular disease includes, but is not limited to, hypertrophy, dilated cardiomyopathy, heart failure, myocardial infarction, hypertension, or stroke.

In one embodiment, the homozygous mutant phospholamban may be associated with early lethality. Identifying and screening families of affected individuals with the identified phospholamban mutation for genetic linkage will provide important insights into identification of this polymorphism as a risk factor for the development of heart failure. Alterations in phospholamban, which has been shown to be a major regulator of myocardial contractility, may contribute to the phenotype of the disease.

The methods will be more fully understood in view of the examples.

### EXAMPLES

Written informed consent is obtained from all participants in accordance with the guidelines of Onassis Cardiac Surgery Center Research Committee. Family members are evaluated by history taking, physical examination, and two-dimensional echocardiography, under normal and stressed conditions, by study personnel who had no knowledge of family member's genotype status. Familial dilated cardiomyopathy is diagnosed. The clinical status of family members who have died is determined on the basis of medical record and postmortem examination.

### Example 1

This example demonstrates a method for phospholamban assessment in an individual. Eighty-six dilated cardiomyopathy (DCM) patients, with New York Association Class I, II, III and IV heart failure with an average age of onset of 44.4±12.3 years, are enrolled in this study from the Onassis Cardiac Surgery Center in Athens, Greece. In addition, thirty unrelated, age matched normal subjects with no known medical illness, family history of heart failure, or use of medication, served as controls.

A number of patients have undergone heart transplantation and some are on the waiting list. An overview of their medical history indicated that all patients are taking at least one form of medication on a continuous basis (ACE inhibitors, diuretic Lanoxin, beta blocker, CCB, anti-arrhythmics, nitrates/hydralazine). Their average ejection fraction was 22.9±9.91%. Some of the patients are diagnosed with hypertension, diabetes, hypercholestrolemia, atrial fibrillation, or thyroid condition. Vesicle coronary artery disease, hypokinesis, and ventricular hypertrophy are common among most of the patients.

In order to identify naturally occurring mutation(s) in the phospholamban complex, which may contribute to the abnormal Ca²⁺ homeostasis of the affected individuals with heart failure, the coding region of phospholamban the coding region is amplified by polymerase chain reaction (PCR) methodology, using isolated genomic DNA from blood samples and analyzed by sequencing.

The phospholamban coding region sequence analysis of eighty-six DCM patients from the Onassis Cardiac Surgery Center in Greece show a T-to-G transversion in nucleic acid position 116 coding region of phospholamban, as illustrated in FIG. 1. As illustrated in FIG. 1, part of the phospholamban gene from proband III-4 (top) reveals the homozygous transversion of T to G in nucleic acid position 116 in the coding sequence for phospholamban, when compared with normal (middle). This point mutation converts the codon for Leu³⁹ (TTA) to a stop codon (TGA), truncating the protein near the center of the hydrophobic transmembrane sequence, which extends from Leu³¹ to Leu⁵². This mutation also deletes a cleavage site for the restriction endonuclease Tru9 I. Nine individuals in the family are heterozygous for the T to G mutation (bottom). The coding region is amplified from 60 ng of genomic DNA using PCR methodology and sequenced. The sequences are compared to the reported human phospholamban sequence. The T-to-G transversion is indicated by arrow in the affected individual.

The presence of a single point mutation in nucleic acid number 116, corresponding to codon 39, converted the amino acid leucine (TTA) to a stop codon (TGA) in the hydrophobic transmembrane domain of phospholamban. Of the seventy-six DCM patients, 2 individuals (2.6%) are identified as carriers of the T-to-G mutation. One affected individual, being heterozygous for the mutated allele, has both a T and a G nucleotide at the same position, while the other individual is homozygous for the mutation. This mutation removes a restriction site for the endonuclease Tru9 I. As illustrated in FIG. 2, analysis of inheritance of the Leu39 (L39)stop mutation based on the Tru 9 I shows restriction endonuclease digestion patterns of PCR amplified DNA fragments from normal and affected individuals. PCR products (15μl) were digested with 1 unit of Tru 9 I and fractionated in a 2% agarose gels. Following digestion, four fragments of 147, 87, 61 and 53 bp are observed in the normal phospholamban product, three fragments of 234, 61, and 53 bp are observed in the homozygous mutant product and five fragments of 234, 147, 87, 61 and 53 bp are observed in the heterozygote. This diagnostic technique is used for further characterization of affected individuals and normal subjects. Thirty normals from the same ethnic background and at similar ages are screened, and none of these carried the mutation.

Blood from normal subjects and patients with heart failure is drawn in sodium citrate and analyzed. Genomic DNA is isolated either from whole blood (leukocyte cells) using a DNA isolation kit (Qiagen) or from paraffin blocks containing heart tissue (patient III-6, deceased at age 17). A 348 base pair fragment of the phospholamban gene containing the phospholamban coding region is amplified using 60 ng of genomic DNA and the polymerase chain reaction (PCR) method. The sequences of the sense and antisense primers are as follows: primer 1 ⁵′TCTCATATTTGGCTGCC³′ (SEQ ID NO:2) and primer 2 ⁵′ATTGTTTTCCTGTCTGC³′ (SEQ ID NO:3). To detect heterozygous individuals, the primers are tagged with M13 forward and reverse primers. The conditions for PCR amplification using Taq polymerase, include the following: one cycle at 94° C. for 3 min linked to 30 cycles of 94° C. for 1 min, 46° C. for 1 min and 72° C. for 1 min followed by one cycle of 94°C. for 1 min, 53°C. for 1 min and 72°C. for 10 min extension. PCR amplified products are separated by electrophoresis on a 1.2% agarose gel and the 348 bp fragment is gel purified using the Qiaquick gel extraction kit (Qiagen). This phospholamban fragment is then sequenced directly using M13 forward and reverse primers. The generated sequences are compared with the reported human phospholamban sequences. For rapid screening, the PCR products are digested with Tru9 I. The reaction products are then subjected to 2% agarose gel electrophoresis and visualized with ethidium bromide.

### Example 2

This example demonstrates the in vitro coexpression of SERCA2a and mutant phospholamban. Mutagenesis of phospholamban cDNA is carried out. To determine whether the T-to-G transversion mutation in the transmembrane domain of the phospholamban coding region affected the activity of SERCA2a, the inventors initiate in vitro studies by co-expressing the rabbit or human wild type or mutant phospholamban constructs with SERCA2a in a 1:1 ratio within HEK-293 cells. Microsomes are isolated from transfected cells and the Ca²⁺ dependence of Ca²⁺ transport is measured for each isolate, as illustrated in FIG. 3 (where (▴) represents SERCA2a alone; (●) represents SERCA2a coexpressed with PLN N27; (▪) represents SERCA2a coexpressed with PLN K27; (▾) represents SERCA2a coexpressed with PLN N27/L39Stop; (♦) represents SERCA2a coexpressed with PLB K27/L39stop).

The reduction in the apparent affinity of SERCA2a for Ca²⁺ (K_{Ca} expressed in pCa units) provides a measure of the inhibitory function of phospholamban. When wild type phospholamban is co-expressed with SERCA2a, a significant rightward shift in the curve of Ca²⁺ dependence of the Ca²⁺ uptake activity is observed, indicating that the interaction of phospholamban with SERCA2a causes a lower apparent affinity for Ca²⁺. However, co-expression of SERCA2a with two different mutant forms of PLN (L39Stop with N27, the rabbit isoform; or L39Stop with K27, the human isoform) has no effect on Ca²⁺ uptake activity (FIG. 3A). These results indicate either that the truncated phospholamban protein could not interact with SERCA2a to inhibit the SERCA2a pump or that the mutant protein is not expressed. Further analysis reveals that the truncated form of phospholamban is not expressed stably in HEK-293 cells (FIG. 3B) Briefly, a 172 bp fragment containing the coding sequence for either rabbit or human phospholamban (bases-6 to 162) is amplified in a recombinant polymerase chain reaction using primers with 5′-add-on sequences containing restriction endonuclease sites for Xba I (5′ end) and Sal I (3′ end). The product is subcloned into pBluescript KS⁺ (Stratagene) after digestion with Xba I and Sal I. Wild type and mutant phospholamban cDNAs are ligated into the Xba I and Sal I sites of the pMT2 expression vector for amplification, and the plasmid DNA is purified using Qiagen columns. Phospholamban and SERCA2a cDNA in the pMT2 vector are cotransfected (8 μg of each cDNA per dish) into HEK-293 cells using the calcium phosphate precipitation method. Cells are harvested 48 h after transfection, and microsomes are prepared and assayed for Ca²⁺ transport activity and the data is analyzed as described previously.

### Example 3

This example demonstrates quantitative immunoblotting of cardiac homogenates to determine levels of phospholamban and SR calcium handling proteins. To determine whether the mutated phospholamban is present in the patient's III-4 explanted heart, quantitative immunoblot analysis is performed. Analysis of the cardiac homogenate reveals the absence of phospholamban protein in the patient carrying the Leu39 to stop codon mutation, consistent with the in vitro co-expression studies. However, phospholamban is present in another heart failure patient without the mutation and its levels are similar to those found in normal subject controls, in agreement with previous observations. Assessment of the SERCA2a protein levels in the same hearts indicates significant decreases (˜50%) in both III-4 and another heart failure patient (without the phospholamban mutation) with no changes in calsequestrin protein levels, as illustrated in FIG. 5.

Specifically, FIG. 5 illustrates quantitative immunoblotting of phospholamban and SERCA2a from explanted heart tissue of homozygous proband III-4. Proteins in cardiac homogenates are subjected to 13% SDS-PAGE, transferred to nitrocellulose membranes and stained with specific antibodies against PLB, SERCA2 and CSQ under conditions where the protein levels of SERCA2a, PLB and CSQ can be quantified. PLN_{H}: PLN pentamer. CSQ protein levels are used to normalize for loading conditions.

### Example 4

To determine the mode of inheritance of the phospholamban mutation in the patient carrying the L39Stop codon mutation, blood samples from family members are collected and their phospholamban genomic sequences are analyzed. The pedigree analysis, as illustrated in FIG. 4, consists of 30 Greek family members extending through four generations. Specifically, as illustrated in FIG. 4, (▭) represents Male, normal; (∘) represents female, normnal; (▪), represents homozygous for the T116 to G mutation; (●) represents female, homozygous for the T116 to G mutation; ( [symbol] ) represents male, heterozygous for the T116 to G mutation; ( [symbol] ) represents female, heterozygous for the T116 to G mutation.

Ten individuals are affected, of which 8 are heterozygous carriers for the Leu 39 mutation. Two siblings (III-6, III-4: brother and sister) from generation III are homozygous carriers for the phospholamban mutation. The homozygous male sibling died at age 17 after cardiac transplantation and the homozygous female sibling underwent cardiac transplantation at the age of 28 in 2002. The heterozygous individuals do not show any detectable clinical phenotype. However, the inter-ventricular septum (IVS: 10, 11, 13 and 13 mm) and posterior wall thickness (PW: 10, 10, 11 and 12.7 mm) are increased in four heterozygous family members, indicating mild hypertrophied hearts in these individuals.

Clinical investigation by echocardiography demonstrates chamber dilation (left ventricular end diastolic dimension, LVEDD: 63 mm, left ventricular end systolic dimension, LVESD: 53 mm) and reduced cardiac function (left ventricular ejection fraction, LVEF: 20%) in the patient, who is homozygous carrier of the Leu39 to stop codon mutation. However, family members, heterozygous for the mutation, do not show any cardiac dysfunction up to the present time. In homozygous family members, the onset of the disease occurs at the ages of 17 year old for the male and 20 year old for the female patient. Patients III-4 (female) and III-6 (male) are hospitalized with heart failure at ages 17 and 28, respectively, and underwent cardiac transplantation for end stage heart failure within several months of hospitalization.

### Example 5

To further assess the dilated cardiomyopathy alterations in the mutant phospholamban hearts, histological analysis is performed on the explanted hearts of the transplanted sibling patients, carrying the Leu39 to stop codon mutation. There is abundant interstitial fibrosis and myofibril disarrangement in both III-6 and III-4 patients' hearts, as illustrated in FIG. 6.

Hearts from patients with mutant phospholamban are subjected to histological analysis. Briefly, heart tissue samples are collected from patients during heart transplantation, fixed overnight in 10% formalin, buffered with PBS, dehydrated in 70% ethanol, transferred to xylene and then into paraffin. Paraffin-embedded heart samples are sectioned at 4 μm and stained with Masson's Trichrome to illustrate the massive interstitial fibrosis and myocardial disarrangement that accompanies DCM.

The specific embodiments in the examples described herein are illustrative in nature only and are not intended to be limiting of the claimed compositions and methods. Additional embodiments and variations within the scope of the claimed invention will be apparent to those of ordinary skill in the art in view of the present disclosure.

What we claim is:

1. A method of phospholamban polymorphism assessment in an individual comprising the steps of:
a. obtaining a sample from the individual;
b. isolating a nucleotide fragment containing a phospholamban coding region from the sample;
c. analyzing the nucleotide fragment; and
d. comparing the analysis of the nucleotide fragment with a predetermined phospholamban nucleotide fragment sequence to determine whether the individual carries a phospholamban polymorphism.

2. The method according to claim 1, wherein the sample comprises blood sample, tissue sample, or combinations thereof.

3. The method according to claim 1, wherein the step of analyzing the nucleotide fragment comprises the steps of:
a. amplifying the nucleotide fragment;
b. purifying the amplified nucleotide fragment; and
c. sequencing the purified nucleotide fragment.

4. The method according to claim 1, wherein the step of analyzing the nucleotide fragment comprises the steps of:
a. amplifying the nucleotide fragment;
b. purifying the amplified nucleotide fragment; and
c. subjecting the amplified nucleotide fragment to restriction endonuclease enzyme analysis.

5. The method according to claim 1, wherein the individual has or is at risk for developing cardiovascular disease.

6. The method according to claim 5, wherein the cardiovascular disease comprises hypertrophy, dilated cardiomyopathy, heart failure, myocardial infarction, hypertension, stroke, or combinations thereof.

7. The method according to claim 1, wherein the phospholamban polymorphism is in the homozygous form.

8. The method according to claim 1, wherein the phospholamban polymorphism is in the heterozygous form.

9. The method according to claim 7, wherein the homozygous form of the phospholamban polymorphism comprises a change of nucleotide acid 116 from nucleic acid T to nucleic acid G in both alleles.

10. The method according to claim 8, wherein the heterozygous form of the phospholamban polymorphism comprise a change of nucleotide 116 from nucleic acid T to nucleic acid G in one allele.

11. The method according to claim 9, wherein the homozygous form of the phospholamban polymorphism comprises a change in codon 39 from a Leucine codon to a stop codon in both alleles.

12. The method according to claim 10, wherein the heterozygous form of the phospholamban polymorphism comprises a change in codon 39 from a Leucine codon to a stop codon in one allele.

13. The method according to claim 11, wherein the homozygous form of the phospholamban polymorphism comprises removal of a restriction endonuclease site in both alleles.

14. The method according to claim 12, wherein the heterozygous form of the phospholamban polymorphism comprises removal of a restriction endonuclease site in one allele.

15. An isolated phospholamban polymorphism fragment comprising SEQ ID NO:1.

16. A method for determining if an individual is at risk for developing a cardiovascular disease, comprising
a. obtaining a sample from the individual;
b. isolating a nucleotide fragment containing a phospholamban coding region from the sample;
c. analyzing the nucleotide fragment; and
d. comparing the analysis of the nucleotide fragment with a predetermined phospholamban nucleotide fragment sequence to determine whether the individual carries a phospholamban polymorphism characteristic of an increased risk of developing cardiovascular disease.

17. A method for determining if an individual is at risk for developing a cardiovascular disease, comprising analyzing a sample from the individual to determine the presence of a phospholamban polymorphism characteristic of an increased risk of developing cardiovascular disease.


SEQUENCE LISTING (6 sequences)

SEQ ID NO: 1 - DNA, 159 bp, Human
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 2 - DNA, 17 bp, human
tctcatatttggctgcc

SEQ ID NO: 3 - DNA, 17 bp, human
attgttttcctgtctgc

SEQ ID NO: 4 - DNA, 14 bp, human
ctcatctgaatatg

SEQ ID NO: 5 - DNA, 14 bp, human
ctcatcttaatatg

SEQ ID NO: 6 - DNA, 13 bp, human
tcatctgaatatg